(19) United States
(12) Patent Application Publication
(10) Pub. No.: US 2024/0350310 A1
(43) Pub. Date: Oct. 24, 2024

(54) CONTROL OF IRRIGATION IN A PHACOEMULSIFICATION SYSTEM

(71) Applicant: JOHNSON & JOHNSON SURGICAL VISION, INC., Irvine, CA (US)

(72) Inventors: Eran Haas, Haifa (IL); Vadim Gliner, Haifa (IL); Adam Walter Toner, Ladera Ranch, CA (US)

(21) Appl. No.: 18/136,532

(22) Filed: Apr. 19, 2023

(51) Int. Cl. A61F 9/007

(52) U.S. Cl. CPC A61F 9/00745; A61F 2009/00887

(57) ABSTRACT

A phacoemulsification system, comprising: a handpiece comprising a piezoelectric element and a needle, the needle configured to be inserted into an eye and to be vibrated by the piezoelectric element to emulsify a lens of the eye; an irrigation module configured to supply a flow of irrigation fluid into the eye; an aspiration module configured for removing material from the eye; circuitry configured to determine whether at least a distal tip of the needle is inside the eye or is outside the eye; and a processor, which is configured to receive a determination from the circuitry as to whether the distal tip of the needle is inside or outside the eye, and to control operation of at least the irrigation module according to the determination.

### TECHNOLOGICAL FIELD

The present application relates to controlled operation of a phacoemulsification system, and more specifically to automated control of irrigation in a phacoemulsification system.

### BACKGROUND

US20220192878A1 discloses “A phacoemulsification system includes a phacoemulsification probe, an irrigation pump, an aspiration pump, circuitry, and a processor. The probe includes a piezoelectric crystal configured to vibrate at a mechanical resonance frequency of the crystal; a needle configured for insertion into an eye and be vibrated by the crystal to emulsify a lens of the eye; an irrigation channel for receiving irrigation fluid from an irrigation line and flowing the irrigation fluid into the lens capsule; and an aspiration channel for removing material from the lens capsule and evacuating the removed material to an aspiration line. The irrigation and aspiration pumps are coupled with the irrigation and the aspiration line, respectively. The circuitry is configured to measure electrical impedance of the crystal. The processor is configured to receive the measured electrical impedance, and to control an operation of the irrigation pump and the aspiration pump according to the measured electrical impedance.”

### OVERVIEW

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then, a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation fluid (e.g. a balanced salt solution) to maintain the anterior chamber of the eye. After removing the cataract with phacoemulsification, the softer outer lens cortex is removed with suction. An intraocular lens is then introduced into the empty lens capsule restoring the patient's vision.

During the phacoemulsification procedure, the eye is irrigated with a balanced salt solution to replace liquid and lens particles that are aspirated from the eye. The rate of irrigation should closely correspond to the rate of aspiration in order to prevent either under-pressure or over-pressure in the eye, either of which can cause trauma. It is noted that during the emulsification of the lens of an eye, eye fluid may comprise natural eye fluid, irrigation fluid, and lens material (e.g., the emulsified parts of the cataract).

The present disclosure relates to identifying a needle position with respect to the eye, e.g., whether the needle is within the eye or is outside the eye and controlling operation of the phacoemulsification system accordingly.

Commonly, irrigation is provided during the phacoemulsification procedure for one or more of: balancing the aspiration of fluid and/or matter, maintaining the pressure inside the eye within prespecified safe limits; facilitating the removal of lens particles from the eye; and cooling the probe. In certain situations, irrigation may continue to flow even when the needle is no longer in the eye (for example if the needle was removed from the eye or has slipped out), potentially causing an unwanted spill and a disturbance to the physician. In some cases, in which irrigation is activated via a foot pedal, it may take some time to the physician to remove their foot from the pedal, and irrigation may continue to flow undesirably, at least until the physician lifts their foot. A system as described in the present disclosure attempts to solve these issues by automated detection of a position of the needle with respect to the eye, namely, determining if the distal tip of the needle is inside or outside the eye. Based on such determination, irrigation can be automatically and immediately controlled, for example stopped if the needle is determined to be outside the surgical site; and/or enabled only when the needle is determined to be inside the surgical site. In some examples, other system functions such as aspiration and vibration of the piezoelectric element (and of the needle as a result) are controlled based on the determination of the needle tip being inside or outside the eye. In an example, aspiration and/or vibration are stopped, (optionally in addition to stopping irrigation), if the needle tip is determined to be outside the eye, when such functions would no longer be of use.

Various methods can be used for determining whether the needle is inside or outside the eye. In a first exemplary method, electrical properties such as impedance of the piezoelectric element are monitored; based on changes in the impedance and/or based on absolute impedance values measured, the system processor can determine whether the needle is inside the eye or is outside the eye. In a second exemplary method, intraocular pressure (IOP) levels or data indicative thereof are monitored; based on changes in IOP and/or based on absolute IOP values, the system processor can determine whether the needle is inside the eye or is outside the eye. In some cases, to avoid modifying the irrigation in response to minor transient changes occurring during procedure, the determination of the needle being inside or outside the eye is reached only after a steady reading is obtained, in which the IOP levels and/or impedance levels are within a predefined range (or over/under a predefined threshold) for a predefined minimal period of time.

As referred to herein, a “needle” may refer to an elongate part configured at a distal portion of the phacoemulsification probe which is intended to be at least partially inserted into the eye. A sleeve can be located coaxially around at least a portion of the needle.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 is flowchart of a general method of controlling irrigation in a phacoemulsification system based on whether a needle of the probe is inside or outside the eye, according to examples of the present disclosure;

FIG. 2 is a schematic, pictorial view of an exemplary phacoemulsification system in which irrigation can be controlled based on whether a needle of the probe is inside or outside the eye, according to examples of the present disclosure;

FIG. 3 is a flowchart of methods for controlling irrigation based on one or both of electrical properties of the piezoelectric element and intraocular pressure (IOP), according to examples of the present disclosure; and

FIG. 4A-B are graphic examples of controlling irrigation based on impedance of the piezoelectric element (FIG. 4A) and based on IOP (FIG. 4B), according to examples of the present disclosure.

### DETAILED DESCRIPTION OF EXAMPLES

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “computing”, “comparing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, a Fabrication Process Examination Information (FPEI) system and respective parts thereof disclosed in the present application.

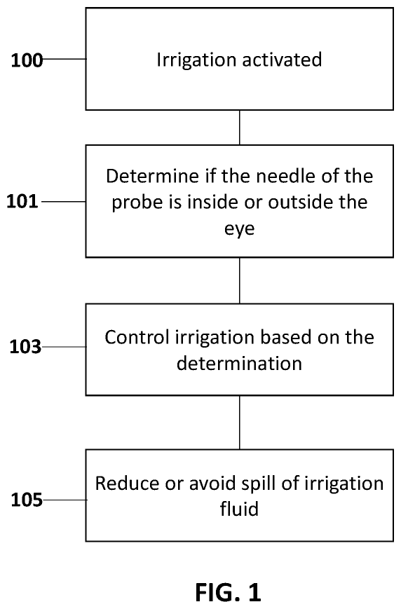

FIG. 1 is a flowchart of a general method for controlling irrigation in a phacoemulsification system.

At 100, irrigation is activated. Activation of irrigation can be achieved, for example, via a foot pedal of the system, for example by pressing the foot pedal into a first position (FP1); via control means (e.g., buttons) on the phacoemulsification handpiece; via the user interface of the phacoemulsification system; and/or otherwise activated.

At 101, a determination is made as to whether the needle, or at least a distal tip thereof, is inside the eye, and/or whether the needle or at least the needle tip is outside the eye, for example if the needle had been removed from the eye or has slipped out or otherwise moved away from the eye.

At 103, the flow of irrigation fluid is controlled based on the determined position of the needle. In some cases, irrigation is stopped when the needle is determined to be outside the eye. Additionally or alternatively, irrigation is enabled (i.e. the flow of irrigation fluid can be provided) only when the needle is determined to be inside the eye.

In some cases, a user (e.g. a physician) can select system settings such as one or more of: automatically stopping irrigation if the needle is determined to be outside the eye; enabling irrigation only if the needle is determined to be inside the eye; selecting a time delay from the determination of the needle position to the actual stop of irrigation flow (for example, allow 0.5 second, 1 second, 2 seconds or intermediate, longer or shorter time period of flow before shutting the flow); and/or other operational settings.

Controlling irrigation in a manner that takes into account whether the needle is currently inside the eye or is currently outside the eye may be advantageous in that a spill of irrigation fluid, which potentially occurs when the needle is outside the eye, can be reduced or avoided, as indicated at 105.

In some examples, a phacoemulsification system is configured to operate in a stabilizing mode in which irrigation is supplied for balancing the aspiration of fluid and/or matter in order to maintain the pressure inside the chamber of the eye within safe limits. In such systems, irrigation can be provided or increased automatically in response to certain flow and/or pressure and/or vacuum indications, e.g., as sensed by one or more sensors of the system. Control of irrigation according to the present disclosure may include overriding this mode of operation, for example as follows: if the needle is removed or otherwise exits the eye, vacuum or low pressure conditions may be sensed, potentially triggering an automated increase in irrigation flow (which, when the needle is in the eye, is desirable for balancing the aspiration). In such case, detecting that the needle is outside the eye and stopping the irrigation flow can prevent a spill or overflow of irrigation fluid.

It is noted that in some examples, irrigation and aspiration are both provided via a co-axial handpiece of the phacoemulsification system (e.g., via two separate channels of the handpiece). In other examples, an additional chamber maintainer device is used with a co-axial handpiece, for providing additional irrigation which maintains the eye shape and pressure. In yet other examples, the handpiece of the phacoemulsification system is configured for aspiration alone, and a separate irrigation cannula provides irrigation. In yet other examples, the handpiece of the phacoemulsification system is configured for aspiration alone, a separate irrigation cannula provides irrigation, and an additional chamber maintainer device is used for providing additional irrigation which maintains the eye shape and pressure.

In examples in which an add-on device such as a cannula or a chamber maintainer are used in addition to the phacoemulsification handpiece, control of irrigation may involve stopping irrigation via the add-on device when determining that the needle of the handpiece is outside the eye; and/or enabling irrigation through the add-on device only when it is determined that the needle of the handpiece is inside the eye.

Irrigation via the add-on device can be controlled in accordance with the determination of whether the needle of the handpiece is inside the eye or is outside the eye. In some examples, irrigation via an add-on device is controlled in a similar manner to irrigation provided via the handpiece, for example, irrigation through both the handpiece and the add-on device would be stopped at the same time. Alternatively, irrigation via the add-on device is controlled in a manner that is set with respect to irrigation provided via the handpiece, for example, first irrigation via the handpiece is stopped and only after a predetermined time period (e.g., several milliseconds) irrigation via the add-on device is stopped, or vice versa: irrigation via the add-on device is stopped and then irrigation via the handpiece is stopped.

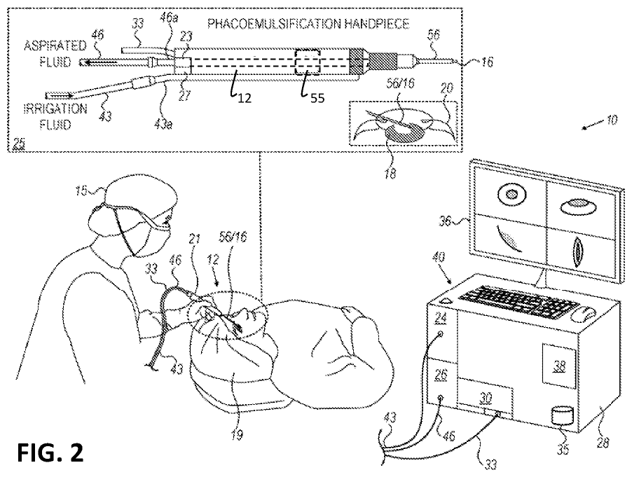

Generally, a phacoemulsification system includes a probe or a handpiece which comprises a needle that is at least partially insertable into an eye; a transducer such as a piezoelectric element that is configured to vibrate the needle for breaking the cataract into smaller particles; an aspiration module which aspirates the particles and withdraws them away from the eye; an irrigation module which introduces fluid into the eye (for one or more of: (i) compensating for eye fluid aspirated from eye for maintaining constant intraocular pressure (IOP) in eye, (ii) controlling a constant temperature in eye (e.g., by dissipating heat generated by the vibration of needle during the procedure, and (iii) facilitating the removal of lens particles from the eye); control means (e.g. a processor); and circuitry (for example sensing circuitry which comprises one or more sensors). A schematic, pictorial view of an exemplary phacoemulsification system in which irrigation can be controlled based on whether a needle of the probe is inside or outside the eye, according to examples of the present disclosure, is shown in FIG. 2.

As seen in the pictorial view of phacoemulsification system 10, and in inset 25, phacoemulsification probe 12 (also referred to herein as a handpiece 12) comprises a needle 16 substantially surrounded by an irrigation sleeve 56. In some examples, needle 16 is hollow and its lumen can be used as an aspiration channel.

Needle 16 is configured for insertion by a physician 15 into an eye 20 of a patient 19, for example into a lens capsule 18 of the eye, to remove a cataract. The needle 16 (and irrigation sleeve 56) are shown in inset 25 as a straight object. However, any suitable needle may be used with phacoemulsification probe 12, for example, a curved or bent tip needle commercially available from Johnson & Johnson Surgical Vision, Irvine, CA., USA.

Referring now to the irrigation and aspiration modules, irrigation channels 43a and aspiration channel 46a are coupled with irrigation tube 43 and aspiration tube 46, respectively.

Referring now to the system circuitry, and more specifically to sensing circuitry, in the shown example, probe 12 includes a sensor 27 coupled with irrigation channel 43a, and a sensor 23 coupled with aspiration channel 46a. It is noted that other examples can include only one sensor in either of the channels. Generally, one or more sensors can be located at any suitable position along the handpiece, and/or operably attached to the handpiece, such as at a module attached at a proximal end of the handpiece.

Sensors 23 and 27 may be any sensor known in the art, including, but not limited to a vacuum sensor or flow sensor. The sensor measurements (e.g., pressure, vacuum, and/or flow) may be taken close to the distal end of the handpiece where the irrigation outlet and the aspiration inlet are located, so as to provide processor 38 an accurate indication of the actual measurements occurring within an eye and provide a short response time to a control loop comprised in processor 38. It is noted that other types of sensors such as temperature sensors, optical sensors, capacitance sensors and/or other sensors may also be incorporated as part of the circuitry of the system.

In some examples, system 10 comprises a processor-controlled irrigation pump 24. During the phacoemulsification procedure, processor-controlled pump 24 comprised in a console 28 pumps irrigation fluid from an irrigation reservoir or tank via irrigation sleeve 56, to irrigate the eye. The fluid is pumped via irrigation tubing line 43 running from console 28 to probe 12. Irrigation pump 24 may be any pump known in the art, for example, a peristaltic pump or a progressive cavity pump. In some examples, a gravity fed irrigation source such as a balanced salt solution bottle or bag may be used with pump 24 or in replacement of the pump.

In some examples, processor 38 controls a pump rate of irrigation pump 24, for example to maintain intraocular pressure within prespecified limits, to enable or disable irrigation, to increase or decrease a flow volume of irrigation fluid, to open or close valves along the irrigation line, or otherwise control irrigation.

In some examples, the processor controls irrigation according to a determination of the needle being inside or outside the eye. Such determination can be reached using measurements obtained by the system circuitry such as readings of pressure received from the sensors (based on which an IOP may be deduced) and/or readings of electrical properties of the piezoelectric element, such as impedance, for example as described hereinabove.

In some examples, eye fluid and waste matter (e.g., emulsified parts of the cataract) are aspirated via hollow needle 16 to a collection receptacle (not shown) by a processor-controlled aspiration pump 26 also comprised in console 28 and using aspiration tubing line 46 running from probe 12 to console 28. In some examples, the same processor that operates the irrigation pump also operates the one or more aspiration pumps, and in some cases, simultaneously. In an example, processor 38 controls an aspiration rate of aspiration pump 26 to maintain intraocular pressure (in case of sub-pressure indicated, for example, by sensor 23) within prespecified limits.

As further shown, phacoemulsification probe 12 includes a piezoelectric element such as a piezoelectric element 55 (e.g., one or more piezoelectric crystals) that drives needle 16 to vibrate, for example to vibrate in a resonant vibration mode that is used to break a cataract into small pieces during a phacoemulsification procedure. Console 28 comprises a piezoelectric drive module 30, coupled with the piezoelectric element, using electrical wiring running in cable 33.

Processor 38 further conveys processor-controlled driving signals via cable 33 to, for example, maintain needle 16 at a selected vibration amplitude. The drive module may be realized in hardware or software, for example, in a proportional-integral-derivative (PID) control architecture.

Processor 38 may receive user-based commands via a user interface 40. Examples of user-based commands may include: setting a vibration mode and/or an operating frequency of the piezoelectric element, setting or adjusting an irrigation and/or aspiration rate of the irrigation pump 24 and aspiration pump 26, respectively, setting or adjusting needle 16 stroke amplitude, turning on irrigation and/or aspiration, turning off irrigation and/or aspiration, and/or otherwise controlling the system.

In some examples, the physician uses a foot pedal (not shown) as a means of control. The foot pedal can be operable at a plurality of different positions, each associated with a different type of system activation. In an example, at an initial (non-stepped) position FP0, no modules are activated; at a first position FP1, irrigation is activated; at a second position FP2, irrigation and aspiration are activated; and at a third position FP3, irrigation, aspiration and vibration of the piezoelectric element are activated. Additionally or alternatively to using a foot pedal, in some systems irrigation is provided (or adjusted, e.g. increased, reduced) in an automated manner, such as in response to activation of aspiration and/or in response to indications obtained by the system sensor(s).

Additionally, or alternatively to the user interface and/or to the foot pedal, processor 38 may receive user-based controls located in a handle 21 of probe 12.

In an example, user interface 40 and display 36 may be integrated into a touch screen graphical user interface. Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35. This software may be downloaded to a device in electronic form, over a network, for example. Alternatively, or additionally, the software may be stored in tangible, non-transitory computer-readable storage media, such as optical, magnetic, or electronic memory.

The system shown in FIG. 2 may include further elements, which are omitted for clarity of presentation. For example, physician 15 typically performs the procedure using a stereo microscope or magnifying glasses, neither of which are shown. Physician 15 may use other surgical tools in addition to probe 12, which are also not shown in order to maintain clarity and simplicity of presentation.

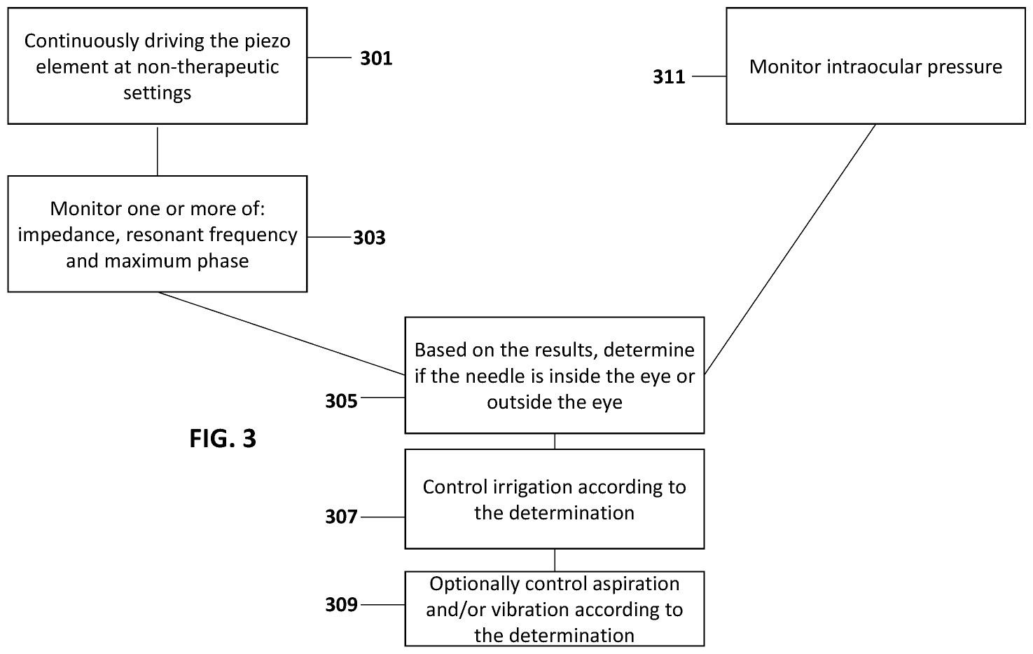

As noted above, the processor can be configured to control the system based on signals received from the system circuitry, for example based on indications from one or more sensors of the system. In some examples, the signals are indicative of a position of the needle with respect to the eye, namely, if the needle is inside or outside the eye. FIG. 3 delineates two examples of automated control of irrigation based on an indication of the needle position obtained by the system circuitry: in the first example, properties of the piezoelectric element serve as indications of the needle position; in the second example, a level of IOP serves as an indication of the needle position.

Referring to the first example, at 301, the piezoelectric element is continuously driven at non-therapeutic settings, for example, at a low power or intensity which does not affect the tissue. Optionally, any movement (if at all) of the needle in response to the activation of the piezoelectric element is insignificant and does not cause emulsification or cavitation.

At 303, one or more properties of the piezoelectric element such as impedance (electrical and/or acoustic), resonant frequency, and maximum phase are monitored. A change in one or more of these properties can be indicative of a current position of the needle (which contains or is connected with or is adjacent the piezoelectric element). The piezoelectric element properties such as the impedance can be measured via the circuitry and/or calculated by detecting the applied voltage and current. In an example, electrical impedance is calculated by driving the piezoelectric element at a certain voltage, measuring the resulting current (optionally by applying discrete Fourier transform of the overall current measured) and then calculating the impedance (voltage divided by the current).

At 305, based on the measured and/or calculated properties of the piezoelectric element, a determination is made as to whether the needle of the probe is inside the eye or is outside the eye. For example, a sudden drop in impedance may indicate that the needle moved from inside the eye to outside the eye (and by that, a mechanical load on the piezoelectric element was reduced or removed, affecting the impedance); a sudden rise in impedance may indicate that the needle has entered the eye. Additionally or alternatively, a low impedance reading (optionally, low impedance values measured over time) may indicate that the needle is outside the eye.

At 307, irrigation is controlled (such as by the system processor) according to the determination. For example, if the needle is determined to be outside the eye, the processor can instruct the irrigation module to halt the flow of irrigation fluid. Optionally, irrigation is stopped immediately following the determination that the needle is outside the eye, for example within 1 second of the needle exiting the eye. In this manner, even if irrigation is still otherwise activated (such as by the user via the foot pedal), the automated stopping of irrigation can be set to override the user activation and halt the flow of fluid to prevent a spill. This may be especially advantageous in case of the needle slipping out of the eye, unintentionally, while the user's foot had not yet been removed from the pedal, continuing to activate the irrigation although that is no longer needed.

At 309, optionally, aspiration and/or vibration of the piezoelectric element are controlled according to the determination of the needle being inside or outside the eye. For example, if it is determined that the needle is outside the eye, the processor may stop aspirating via the aspiration module. For example, if it is determined that the needle is outside the eye, the processor may stop vibrating the piezoelectric element. Such control may be provided in addition to the control of irrigation, or independently thereof.

Referring to the second example, in which a level of IOP serves as an indication of the needle position: at 311 the IOP is monitored. The IOP can be measured continuously, optionally throughout the procedure. Optionally, IOP levels are checked periodically, e.g., every 1 second or a shorter time period. The IOP can serve as an indicator of the needle position since when the needle is inside the eye, generally, a positive level of IOP would be measured; and when the needle is outside the eye, the level of IOP is expected to drop to about 0 mmHg. In some examples, the level of IOP is measured via the system circuitry, for example using one more sensor(s) such as pressure or flow sensors coupled with the irrigation channel and/or the aspiration channel, which during the procedure are positioned at or adjacent the eye, close enough to detect a change in pressure inside the eye. Additionally or alternatively, IOP measurements are obtained by means external to the phacoemulsification system, for example using a manometer, tonometer, or other suitable measurement device. Based on the IOP levels, a determination is made as to whether the needle of the probe is inside the eye or is outside the eye, as described with respect to 305; irrigation is then controlled accordingly, as described above with respect to 307. Optionally, aspiration and/or vibration are also controlled based on the determination, as described above with respect to 309.

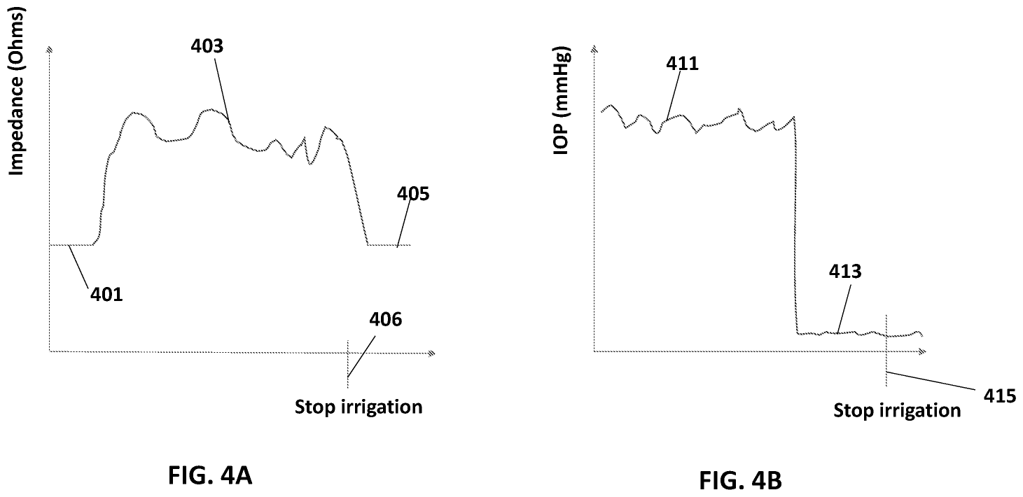

Graphic examples of irrigation control, in accordance with some examples, are shown in FIGS. 4A-B.

FIG. 4A shows an example of changes in impedance of the piezoelectric element over time. At a first segment 401, the piezoelectric element is continuously driven at non-therapeutic settings, such as at a power of 0.05 Watts and a frequency of 39 kHz. The impedance expected using these operation settings is about 1100 Ohms.

Generally, a frequency within the range of 20 kHz to 120 KHz can be used when driving the piezoelectric element at non-therapeutic settings.

Optionally, the continuous activation of the piezoelectric element at non-therapeutic settings can provide a baseline impedance level to which additional impedance values measured during the procedure can be compared. In an example, the baseline impedance level is determined as the level measured before a significant change (e.g., a rise) in impedance which occurs when the needle is introduced into the eye, as demonstrated by second segment 403: when the needle is introduced into the eye, the impedance rises, for example to about 1700 Ohms. This higher impedance is measured during the time period in which the needle is inside the eye. At a third segment 405 of the graph, a drop in impedance occurs, indicating that the needle was removed from the eye or has otherwise exited the eye. Optionally, when the needle is no longer inside the eye, the impedance level returns to the baseline level measured prior to insertion of the needle into the eye; or, if the piezoelectric element is no longer activated, no or zero impedance will be measured.

When it is determined that the needle is no longer inside the eye, irrigation can be stopped (see 406). In some examples, irrigation is stopped when the detected impedance falls below a predetermined threshold. Such predetermined threshold can be selected based on the type of piezoelectric element being used (i.e., on the specific type of the crystal); based on the type of needle used; based on experimentation; based on the specific structure and arrangement of components in the handpiece and/or other factors.

Optionally, irrigation is stopped when a steady reading of impedance is obtained, i.e., the impedance readings are below a predetermined threshold for a minimal period of time, e.g., at least 100 msec, 250 msec, 500 msec or intermediate, longer or shorter time period. Additionally or alternatively, irrigation is stopped when a slope of the change in impedance is detected and/or calculated as exceeding a predetermined threshold or range for the slope, indicating a steady drop.

In some examples, impedance thresholds or ranges are selected so that only a substantial change in impedance will trigger control of irrigation (e.g., stopping of irrigation), while minor changes in impedance, which are likely to occur during the standard procedure, will not affect this control. Determining which change in impedance is substantial enough so as to trigger control of irrigation can be performed based on experimentation, based on the type of piezoelectric element being used, based on the type of needle used, and/or based on the sensitivity of control requested by the user.

It is noted that the expected impedance values, for example the predetermined ranges or thresholds of impedance may change from system to system, depending for example on the specific type and/or size of needle used. In addition, contact of the needle with certain materials inside the eye such as cataract particles (which are relatively hard) may also affect the impedance, but to an extent lesser than a change in position of the needle being inside the eye or outside the eye would.

In some examples, the stopping of irrigation flow is achieved by a processor of the system signaling the irrigation pump to stop the flow of irrigation fluid along the irrigation line and channel; by the processor signaling a valve configured along the irrigation line to close the path; and/or other suitable means for stopping the irrigation flow.

FIG. 4B shows an example of changes in IOP over time (or changes of data indicative of the IOP, i.e., changes over time of an IOP indicator). At a first segment 411 of the graph, the IOP is maintained within a predefined range in which aspiration of fluid and matter from the eye is balanced with incoming flow of irrigation fluid, for maintaining the chamber of the eye intact and keeping the pressure inside the eye within prespecified limits. In an example, the IOP is maintained within a range of 40 mmHg-70 mmHg, 30 mmHg-60 mmHg, 50 mmHg-80 mmHg or intermediate, higher or lower level. At a second segment 413 of the graph, the IOP substantially drops, for example to a level of 0 mmHg or close to 0 mmHg (e.g., 0 +/−15 mmHg, 0+/−20 mmHg, 0+/−10 mmHg or intermediate, higher or lower level). Such drop in the IOP and/or a low IOP level (absolute value thereof) can indicate that the needle has moved away or is no longer inside the eye, triggering the control of irrigation in response. In some cases, irrigation is stopped (see 415) only when the IOP (or IOP indicator) i.e., IOP readings remain steady and low (e.g., zero or close to zero) for a predetermined time period such as 100 msec, 250 msec, 500 msec or intermediate, or longer time periods. In some cases, IOP thresholds or ranges are selected so that only a substantial change in IOP will trigger control of irrigation (e.g., stopping of irrigation), while a transient or minor change in IOP will not trigger such control. In an example, a vacuum surge occurring within the eye could cause transient change in the IOP level, while the needle is still inside the eye. In such situation, irrigation should not be stopped. By awaiting a steady reading of IOP, in which the IOP remains at low or zero levels over time, unintended stopping of irrigation can be avoided, and irrigation would be stopped if and only if the needle is no longer in the eye.

### SUMMARY OF THE PRESENT DISCLOSURE

### Example 1

A phacoemulsification system (10), comprising a handpiece (12) comprising a piezoelectric element (55) and a needle (16), wherein the needle is configured to be inserted into an eye and to be vibrated by the piezoelectric element to emulsify a lens of the eye; an irrigation module configured to supply a flow of irrigation fluid into the eye; an aspiration module configured for removing material from the eye; circuitry configured to determine whether at least a distal tip of the needle is inside the eye or is outside the eye; and a processor (38), which is configured to receive a determination from the circuitry as to whether the distal tip of the needle is inside or outside the eye, and to control operation of at least the irrigation module according to the determination.

### Example 2

The system according to Example 1, wherein the processor is configured for at least one of: (i) stopping the flow of irrigation fluid when the circuitry determines that the needle is outside the eye; or (ii) enabling the flow of irrigation fluid only when the circuitry determines that the needle is inside the eye.

### Example 3

The system according to Example 1 or Example 2, wherein the circuitry is configured to measure intraocular pressure (IOP).

### Example 4

The system according to Example 3, wherein the circuitry is configured to measure IOP via at least one sensor (23,27) incorporated in the handpiece or attached thereto.

### Example 5

The system according to Example 4, wherein the irrigation module is comprised of: an irrigation channel (43a) having an outlet directed into the eye; an irrigation line (43) extending from the irrigation channel; and an irrigation pump (24) which supplies the irrigation fluid into the irrigation line; wherein the at least one sensor (27) comprises a pressure sensor configured at the irrigation channel, adjacent the outlet.

### Example 6

The system according to any of Examples 3-5, wherein the processor is configured to stop the flow of irrigation fluid when the detected IOP is within a predefined range for a minimal predefined period of time.

### Example 7

The system according to Example 6, wherein the predefined range is between 0 mmHg+/−15 mmHg and the minimal predefined period of time is 250 msec.

### Example 8

The system according to any of Examples 1-7, wherein the processor is configured to continuously drive the piezoelectric element at non-therapeutic settings when not emulsifying the lens.

### Example 9

The system according to any of Examples 1-8, wherein the circuitry is configured to measure electrical impedance of the continuously driven piezoelectric element.

### Example 10

The system according to any of Examples 1-9, wherein the processor is configured to stop the flow of irrigation fluid when:
a. a substantial drop in electrical impedance is detected and/or
b. the detected electrical impedance is lower than a preset threshold.

### Example 11

The system according to any of Examples 1-10, wherein the processor is configured to operate the irrigation module and the aspiration module according to a stabilizing mode in which IOP levels are maintained within prespecified safe limits; wherein the processor is further configured so that even in said stabilizing mode, if a determination from the circuitry that the distal tip of the needle is outside the eye is received, irrigation is stopped.

### Example 12

The system according to any of Examples 1-11, further comprising a foot pedal which selectively activates said irrigation module, aspiration module and piezoelectric element via a plurality of foot pedal positions; wherein the processor is configured to control irrigation based on the determination obtained by the circuitry in combination with a current foot pedal position.

### Example 13

The system according to any of Examples 1-12, wherein the processor is configured so that when the circuitry determines that the needle is outside the eye, the processor stops the flow of irrigation fluid even if the foot pedal is at a position which activates irrigation.

### Example 14

The system according to any of Examples 1-13, wherein the processor is configured to control one or both of the piezoelectric element and the aspiration module, in addition to the irrigation module, according to the determination obtained by the circuitry.

### Example 15

The system according to any of Examples 1-14, wherein the processor is configured to stop one or both of aspiration and vibration of the piezoelectric element when the circuitry determines that the needle is outside the eye.

### Example 16

A method of operating a phacoemulsification system (10), comprising: inserting into an eye a at least a part of a handpiece (12) comprising a piezoelectric element (55), a needle (16), an irrigation channel (43a), and an aspiration channel (46a); vibrating the needle to emulsify a lens of the eye; flowing irrigation fluid through the irrigation channel into the eye; removing material from the eye through the aspiration channel; determining, via system circuitry, whether at least a distal tip of the needle is inside the eye or is outside the eye; and via a system processor (38), controlling at least the flow of irrigation fluid based on the determination.

### Example 17

The method according to Example 16, wherein determining comprises measuring or estimating IOP and determining if the needle is inside the eye or outside the eye based on the IOP.

### Example 18

The method according to Example 16 or Example 17, further comprising continuously driving the piezoelectric element at non-therapeutic settings; and determining comprises monitoring impedance of the piezoelectric element and determining if the needle is inside the eye or outside the eye based on the monitored impedance.

As such, those skilled in the art to which the present invention pertains, can appreciate that while the present invention has been described in terms of preferred examples, the concept upon which this disclosure is based may readily be utilized as a basis for the designing of other structures, systems and processes for carrying out the several purposes of the present invention.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing any departure from the scope of the disclosure.

It will also be understood that the system according to the present disclosure may be, at least partly, implemented on a suitably programmed computer. Likewise, the present disclosure contemplates a computer program being readable by a computer for executing the method of the invention. The present disclosure further contemplates a non-transitory computer-readable memory tangibly embodying a program of instructions executable by the computer for executing the method of the present disclosure.

Also, it is to be understood that the phraseology and terminology employed herein are for the purpose of description and should not be regarded as limiting.

It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases.

It is important, therefore, that the scope of the invention is not construed as being limited by the illustrative examples set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. A phacoemulsification system, comprising:
a handpiece comprising a piezoelectric element and a needle, wherein the needle is configured to be inserted into an eye and to be vibrated by the piezoelectric element to emulsify a lens of the eye;
an irrigation module configured to supply a flow of irrigation fluid into the eye;
an aspiration module configured for removing material from the eye;
circuitry configured to determine whether at least a distal tip of the needle is inside the eye or is outside the eye; and
a processor, which is configured to receive a determination from the circuitry as to whether the distal tip of the needle is inside or outside the eye, and to control operation of at least the irrigation module according to the determination.

2. The system according to claim 1, wherein the processor is configured for at least one of:
(i) stopping the flow of irrigation fluid when the circuitry determines that the needle is outside the eye; or
(ii) enabling the flow of irrigation fluid only when the circuitry determines that the needle is inside the eye.

3. The system according to claim 1, wherein the circuitry is configured to measure intraocular pressure (IOP).

4. The system according to claim 3, wherein the circuitry is configured to measure IOP via at least one sensor incorporated in the handpiece or attached thereto.

5. The system according to claim 4, wherein the irrigation module is comprised of: an irrigation channel having an outlet directed into the eye; an irrigation line extending from the irrigation channel; and an irrigation pump which supplies the irrigation fluid into the irrigation line; wherein the at least one sensor comprises a pressure sensor configured at the irrigation channel, adjacent the outlet.

6. The system according to claim 3, wherein the processor is configured to stop the flow of irrigation fluid when the detected IOP is within a predefined range for a minimal predefined period of time.

7. The system according to claim 6, wherein the predefined range is between 0 mmHg+/−15 mmHg and the minimal predefined period of time is 250 msec.

8. The system according to claim 1, wherein the processor is configured to continuously drive the piezoelectric element at non-therapeutic settings when not emulsifying the lens.

9. The system according to claim 8, wherein the circuitry is configured to measure electrical impedance of the continuously driven piezoelectric element.

10. The system according to claim 9, wherein the processor is configured to stop the flow of irrigation fluid when:
a. a substantial drop in electrical impedance is detected and/or
b. the detected electrical impedance is lower than a preset threshold.

11. The system according to claim 1, wherein the processor is configured to operate the irrigation module and the aspiration module according to a stabilizing mode in which IOP levels are maintained within prespecified safe limits; wherein the processor is further configured so that even in said stabilizing mode, if a determination from the circuitry that the distal tip of the needle is outside the eye is received, irrigation is stopped.

12. The system according to claim 1, further comprising a foot pedal which selectively activates said irrigation module, aspiration module and piezoelectric element via a plurality of foot pedal positions; wherein the processor is configured to control irrigation based on the determination obtained by the circuitry in combination with a current foot pedal position.

13. The system according to claim 12, wherein the processor is configured so that when the circuitry determines that the needle is outside the eye, the processor stops the flow of irrigation fluid even if the foot pedal is at a position which activates irrigation.

14. The system according to claim 1, wherein the processor is configured to control one or both of the piezoelectric element and the aspiration module, in addition to the irrigation module, according to the determination obtained by the circuitry.

15. The system according to claim 14, wherein the processor is configured to stop one or both of aspiration and vibration of the piezoelectric element when the circuitry determines that the needle is outside the eye.

16. A method of operating a phacoemulsification system, comprising:
inserting into an eye a at least a part of a handpiece comprising a piezoelectric element, a needle, an irrigation channel, and an aspiration channel;
vibrating the needle to emulsify a lens of the eye;
flowing irrigation fluid through the irrigation channel into the eye;
removing material from the eye through the aspiration channel;
determining, via system circuitry, whether at least a distal tip of the needle is inside the eye or is outside the eye; and
via a system processor, controlling at least the flow of irrigation fluid based on the determination.

17. The method according to claim 16, wherein determining comprises measuring or estimating IOP and determining if the needle is inside the eye or outside the eye based on the IOP.

18. The method according to claim 16, further comprising continuously driving the piezoelectric element at non-therapeutic settings; and determining comprises monitoring impedance of the piezoelectric element and determining if the needle is inside the eye or outside the eye based on the monitored impedance.
